(19) United States
(12) Patent Application Publication
(10) Pub. No.: US 2026/0069304 A1
(43) Pub. Date: Mar. 12, 2026

(54) SURGICAL TOOL END EFFECTORS WITH REPLACEABLE BLADE ASSEMBLIES

(71) Applicant: CILAG GMBH INTERNATIONAL, Zug (CH)

(72) Inventors: Konstantin G. ZABOTKIN, Mason, OH (US); John A. HIBNER, Mason, OH (US); Joseph M. NATH, West Chester, OH (US)

(73) Assignee: CILAG GMBH INTERNATIONAL, Zug (CH)

(21) Appl. No.: 18/826,910

(22) Filed: Sep. 6, 2024

(51) Int. Cl. A61B 17/32; A61B 17/00; A61B 17/3201

(52) U.S. Cl. CPC A61B 17/320016; A61B 17/3201; A61B 2017/00473; A61B 2017/00526

(57) ABSTRACT

A replaceable blade assembly for an end effector of a surgical tool includes a first blade having an inner side and an outer side, the first blade having an open-ended slot that opens in the proximal direction, the open-ended slot of the first blade having an axle opening and an axle slot. The replaceable blade assembly also includes a second blade having an inner side and an outer side, the second blade having an open-ended slot that opens in the proximal direction, the open-ended slot of the second blade having an axle opening and an axle slot. Further, the replaceable blade assembly includes a retainer arranged through the axle openings of the open-ended slots for coupling the first blade and the second blade together, with the inner sides of the first and second blades facing each other, while permitting relative rotation between the first and second blade.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system. Moving the drive cables articulates the end effector to desired angular positions and configurations.

Some end effectors include high-wear components that can mechanically or physically degrade over time and thereby limit the useful life of the end effector. One example of high-wear components is the blades of surgical scissors, which can dull over time, and thereby adversely affect the efficiency of the end effector.

What is needed is a more easily replaceable blade assembly utilizable with an end effector, which can provide a user (e.g., a surgeon, a nurse, etc.) with a new set of blades for every new use of the surgical tool.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is an enlarged isometric view of the distal end of the surgical tool of FIG. 2.

FIGS. 5A and 5B are enlarged front-left and front-right isometric views, respectively, of the end effector of FIG. 4, and FIG. 5C is an enlarged rear-left isometric view of the end effector of FIG. 4, according to one or more embodiments.

FIGS. 6A and 6B are exploded rear-left and front-left isometric views, respectively, of the end effector of FIG. 4, according to one or more embodiments.

FIGS. 7A and 7B are enlarged exploded front-right and rear-left isometric views, respectively, of the removable blade assembly of the end effector of FIG. 4, according to one or more embodiments.

FIG. 8 is an enlarged isometric view of the distal end of the end effector of FIGS. 4-5B without the replaceable blade assembly, according to one or more embodiments of the disclosure.

FIG. 9 depicts an installation of the blade assembly within the end effector, according to one or more embodiments of the disclosure.

FIG. 10 depicts an alternate removable blade assembly, according to one or more embodiments of the present disclosure.

### DETAILED DESCRIPTION

The present disclosure is related to robotic surgical systems and, more particularly, to methods and systems of replacing blades of end effector surgical scissors.

The end effectors described herein can include a blade assembly having opposing first and second blades mounted to first and second blade holders rotatably mounted to an axle. The blade assembly may be snap-fit onto the axle extending between the blade holders and upon (and about) which the blade holders may rotate. The blade assembly may then be pulled off the axle to be removed after a procedure and replaced within a new (and sterile) blade assembly before undergoing a new procedure. Methods of installing and then replacing end effector blades are discussed herein and include moving a blade assembly towards a pair of blade holders of an end effector to thereby installing the blade assembly within the blade holders, and thereafter pulling the blade assembly away from the blade holders and thereby removing the blade assembly from the blade holders.

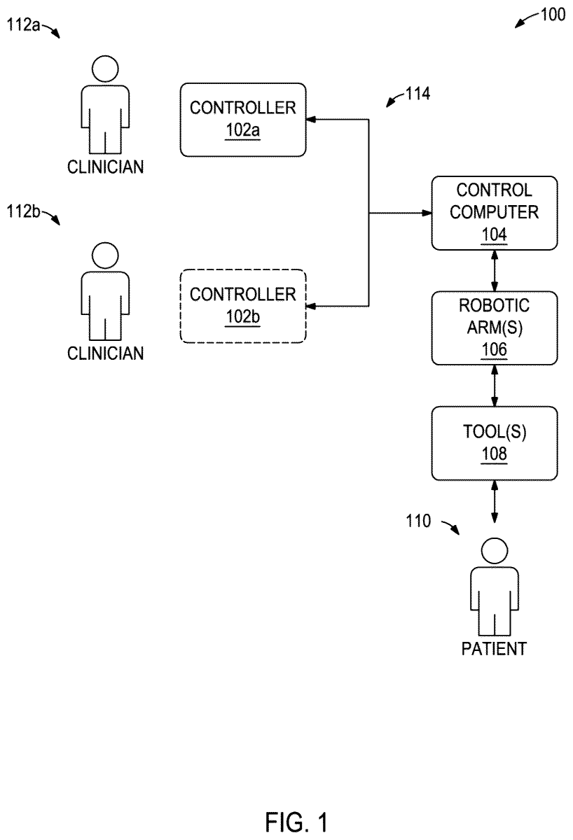

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

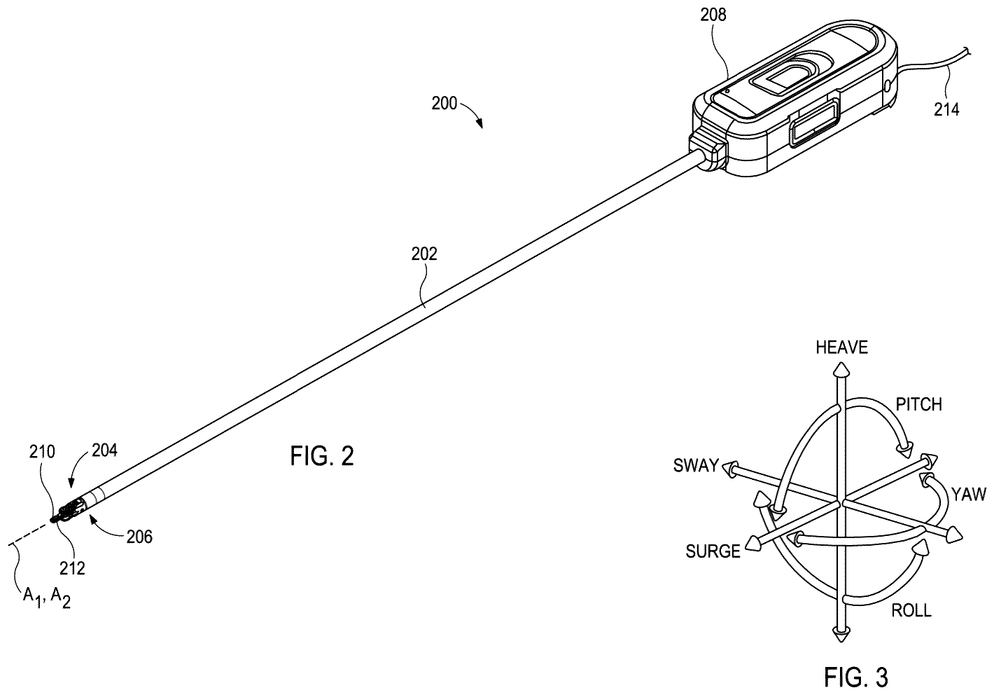

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the robotic surgical system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the drive housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the drive housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A₁ of the shaft 202. In such embodiments, at least one of the drive inputs included in the drive housing 208 is configured to control rotational movement of the shaft 202 about the longitudinal axis A₁.

The shaft 202 is an elongate member extending distally from the housing 208 and has at least one lumen extending therethrough along its axial length. In some embodiments, the shaft 202 may be fixed to the drive housing 208, but could alternatively be rotatably mounted to the drive housing 208 to allow the shaft 202 to rotate about the longitudinal axis A₁. In yet other embodiments, the shaft 202 may be releasably coupled to the drive housing 208, which may allow a single drive housing 208 to be adaptable to various shafts having different end effectors.

The end effector 204 can exhibit a variety of sizes, shapes, and configurations. In the illustrated embodiment, the end effector 204 comprises surgical scissors that includes opposing first (upper) and second (lower) blades 210, 212 configured to move (articulate) between open and closed positions. As will be appreciated, however, the blades 210, 212 may alternatively comprise opposing jaws that form part of other types of end effectors such as, but not limited to, a needle driver, a clip applier, a tissue grasper, a vessel sealer, a combination tissue grasper and vessel sealer, a babcock including a pair of opposed grasping jaws, bipolar jaws (e.g., bipolar Maryland grasper, forceps, a fenestrated grasper, etc.), etc. One or both of the blades 210, 212 may be configured to pivot to articulate the end effector 204 between the open and closed positions.

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw”refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A₁, A₂ would be angularly offset from each other such that the end effector 204 is at a non-zero angle relative to the shaft 202.

Similar to most surgical tools, the surgical tool 200 includes various high-wear components referred to herein as “consumables” that, over time, can mechanically or physically degrade and thereby limit the useful life of the surgical tool 200. Consequently, the surgical tool 200 may be designed to be used for only a predetermined number of procedures. Once the predetermined number of procedures is reached, the operator (e.g., a nurse, a doctor, etc.) may be unable to continue using the surgical tool 200. In such cases, the surgical tool 200 would conventionally be discarded, which can have an adverse impact on the environment.

In some embodiments, the surgical tool 200 may be supplied with electrical power (current) via a power cable 214 coupled to the drive housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator that supplies energy, such as electrical energy (e.g., radio frequency energy), ultrasonic energy, microwave energy, heat energy, or any combination thereof, to the surgical tool 200 and, more particularly, to the end effector 204. Accordingly, the generator may comprise a radio frequency (RF) source, an ultrasonic source, a direct current source, and/or any other suitable type of electrical energy source that may be activated independently or simultaneously.

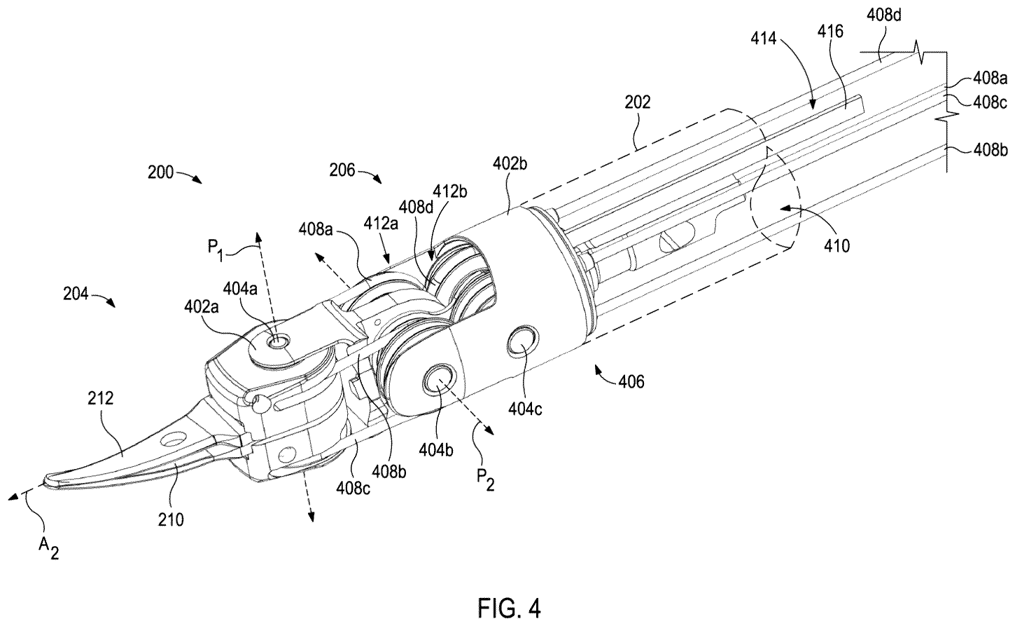

FIG. 4 is an enlarged isometric view of the distal end of the surgical tool 200, according to one or more embodiments. More specifically, FIG. 4 depicts enlarged views of the end effector 204 and the wrist 206, with the end effector 204 in the unarticulated position. The wrist 206 operatively couples the end effector 204 to the shaft 202. To accomplish this, the wrist 206 includes a distal clevis 402a and a proximal clevis 402b. The end effector 204 (i.e., including the blades 210, 212) is rotatably mounted to the distal clevis 402a at a first axle 404a, the distal clevis 402a is rotatably mounted to the proximal clevis 402b at a second axle 404b, and the proximal clevis 402b is coupled to a distal end 406 of the shaft 202.

The wrist 206 provides a first pivot axis P₁ that extends through the first axle 404a and a second pivot axis P₂ that extends through the second axle 404b. The first pivot axis P₁ is substantially perpendicular (orthogonal) to the longitudinal axis A₂ of the end effector 204, and the second pivot axis P₂ is substantially perpendicular (orthogonal) to both the longitudinal axis A₂ and the first pivot axis P₁. Movement about the first pivot axis P₁ provides “yaw” articulation of the end effector 204, and movement about the second pivot axis P₂ provides “pitch” articulation of the end effector 204. In the illustrated embodiment, the blades 210, 212 are mounted at the first pivot axis P₁, thereby allowing the blades 210, 212 to pivot relative to each other to open and close the end effector 204 or alternatively pivot in tandem to articulate the orientation of the end effector 204.

A plurality of drive cables, shown as drive cables 408a, 408b, 408c, and 408d, extend longitudinally within a lumen 410 defined by the shaft 202 and pass through the wrist 206 to be operatively coupled to the end effector 204. While four drive cables 408a-d are depicted in FIG. 4, more or less than four drive cables 408a-d may be included, without departing from the scope of the disclosure.

The drive cables 408a-d form part of the cable driven motion system housed within the drive housing 208 (FIG. 2), and may comprise cables, bands, lines, cords, wires, woven wires, ropes, strings, twisted strings, elongate members, belts, shafts, flexible shafts, drive rods, or any combination thereof. The drive cables 408a-d can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. While four drive cables 408a-d are depicted in FIG. 4, more or less than four may be employed, without departing from the scope of the disclosure.

The drive cables 408a-d extend proximally from the end effector 204 to the drive housing 208 (FIG. 2) where they are operatively coupled to various actuation mechanisms or devices housed (contained) therein to facilitate longitudinal movement (translation) of the drive cables 408a-d within the lumen 410. Selective actuation of all or a portion of the drive cables 408a-d causes the end effector 204 (e.g., one or both of the blades 210, 212) to articulate (pivot) relative to the shaft 202. More specifically, selective actuation causes a corresponding drive cable 408a-d to translate longitudinally within the lumen 410 and thereby cause pivoting movement of the end effector 204. One or more drive cables 408a-d, for example, may translate longitudinally to cause the end effector 204 to articulate (e.g., both of the blades 210, 212 angled in a same direction), to cause the end effector 204 to open (e.g., one or both of the blades 210, 212 move away from the other), or to cause the end effector 204 to close (e.g., one or both of the blades 210, 212 move toward the other).

Moving the drive cables 408a-d can be accomplished in a variety of ways, such as by triggering an associated actuator or mechanism operatively coupled to or housed within the drive housing 208 (FIG. 2). Moving a given drive cable 408a-d constitutes applying tension (i.e., pull force) to the given drive cable 408a-d in a proximal direction, which causes the given drive cable 408a-d to translate and thereby cause the end effector 204 to move (articulate) relative to the shaft 202.

The wrist 206 includes a first plurality of pulleys 412a and a second plurality of pulleys 412b, each configured to interact with and redirect the drive cables 408a-d for engagement with the end effector 204. The first plurality of pulleys 412a is mounted to the proximal clevis 402b at the second axle 404b and the second plurality of pulleys 412b is also mounted to the proximal clevis 402b but at a third axle 404c located proximal to the second axle 404b. The first and second pluralities of pulleys 412a,b cooperatively redirect the drive cables 408a-d through an “S” shaped pathway before the drive cables 408a-d are operatively coupled to the end effector 204.

In at least one embodiment, one pair of drive cables 408a-d is operatively coupled to each blade 210, 212 and configured to “antagonistically” operate the corresponding blade 210, 212. In the illustrated embodiment, for example, the first and second drive cables 408a,b are coupled to (terminate at) the second blade 212, and the third and fourth drive cables 408c,d are coupled to (terminate at) the first blade 210. Actuation of the first drive cable 408a acts on and pivots the second blade 212 about the first pivot axis P₁ toward the closed position. In contrast, actuation of the second drive cable 408b acts on and pivots the second blade 212 about the first pivot axis P₁ toward the open position. Similarly, actuation of the third drive cable 408c pivots the first blade 210 about the first pivot axis P₁ toward the closed position, while actuation of the fourth drive cable 408d pivots the first blade 210 about the first pivot axis P₁ toward the open position.

Accordingly, the drive cables 408a-d may be characterized or otherwise referred to as “antagonistic” cables that cooperatively (yet antagonistically) operate to cause relative or tandem movement of the first and second blades 210, 212. When the first drive cable 408a is actuated (moved), the second drive cable 408b naturally follows since it is also coupled to the second blade 212, and vice versa. Similarly, when the third drive cable 408c is actuated, the fourth drive cable 408d naturally follows since it is also coupled to the first blade 210, and vice versa.

The surgical tool 200 may also include an electrical conductor 414 that supplies electrical energy to the end effector 204, thereby converting the surgical tool 200 into an “electrosurgical instrument”. In other embodiments, however, the electrical conductor 414 may be omitted, and the end effector 204 may operate merely as surgical scissors. In embodiments where the end effector 204 comprises an electrosurgical instrument, however, the electrical conductor 414 extends longitudinally within the lumen 410 and passes through the wrist 206 to be operatively (and electrically) coupled to the end effector 204. In some embodiments, the electrical conductor 414 and the power cable 214 (FIG. 2) may comprise the same structure. In other embodiments, however, the electrical conductor 414 may be electrically coupled to the power cable 214. In yet other embodiments, the electrical conductor 414 may extend to the drive housing 208 (FIG. 2) where it is electrically coupled to an internal power source, such as batteries or fuel cells.

The electrical conductor 414 may include a supply conductor 416 encapsulated by an insulating cover (e.g., an insulated wire). In the illustrated embodiment, the end effector 204 is configured for monopolar operation. Accordingly, electrical energy is transmitted by the supply conductor 416 to the end effector 204, which acts as an active (or source) electrode. In at least one embodiment, the electrical energy may comprise radio frequency (“RF”) energy exhibiting a frequency between about 100 kHz and 1 MHz. Low frequency RF energy causes ionic agitation or friction, in effect resistive heating, thereby increasing the temperature of target tissue. Accordingly, electrical energy supplied to the end effector 204 is converted to heat and transferred to adjacent tissue to cut, cauterize, and/or coagulate the tissue (dependent upon the localized heating of the tissue), and thus may be particularly useful for sealing blood vessels or diffusing bleeding. Electrical energy is then returned from the tissue through a return electrode, which typically comprises a grounding pad separately located on a patient's body.

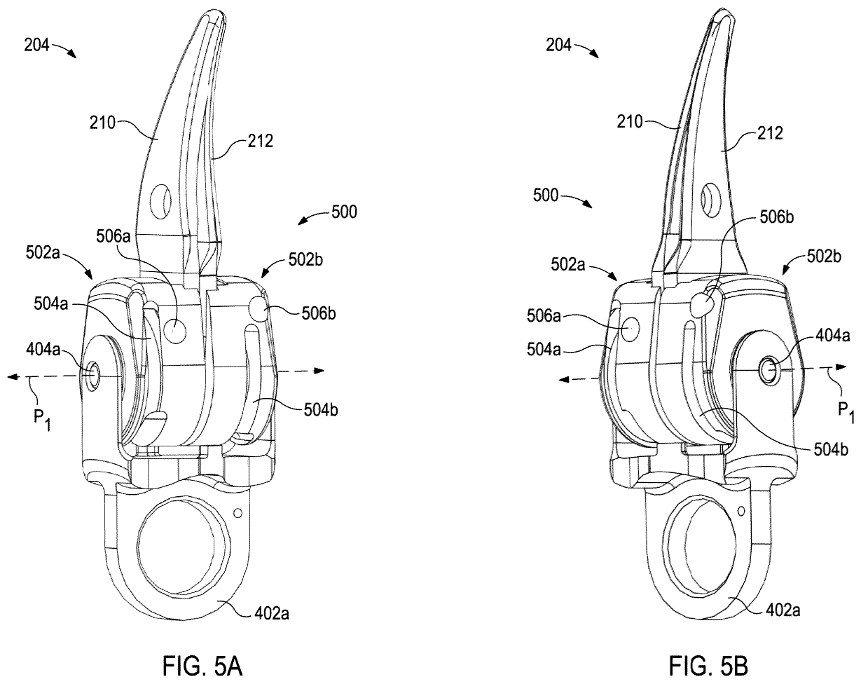

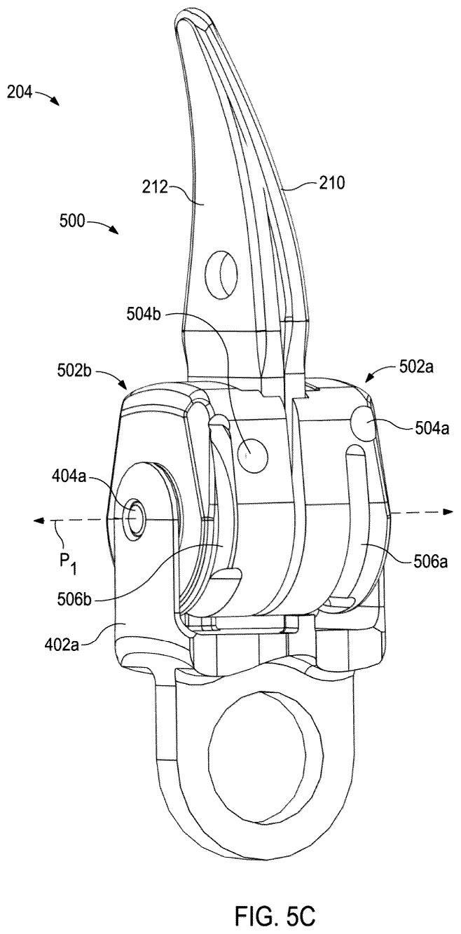

FIGS. 5A and 5B are enlarged front-right and rear-left isometric views, respectively, of the end effector 204 of FIG. 4, according to one or more embodiments. FIG. 5C is an enlarged isometric rear-right view of the end effector 204 of FIG. 4, according to one or more embodiments. As mentioned above, the end effector 204 includes the first and second blades 210, 212 rotatably mounted to the distal clevis 402a at the first axle 404a. The drive cables 408a-d of FIG. 4 are omitted to enable better viewing of the component parts of the end effector 204. Also, while the distal clevis 402a forms part of the wrist 206 and is depicted in FIGS. 5A-5C, other components of the wrist 206 detailed above are omitted from FIGS. 5A and 5B (apart from the distal clevis 402a) for ease of illustration and to enable better viewing of the component parts of the end effector 204.

As illustrated, the end effector 204 further includes first and second blade holders 502a and 502b rotatably mounted to the first axle 404a and laterally offset from each other. The first blade holder 502a is configured to receive and seat the first blade 210 such that movement (rotation) of the first blade holder 502a about the first pivot axis P₁ correspondingly moves (rotates) the first blade 210. The first blade holder 502a may also provide and otherwise define a pair of first tracks or pulleys 504a and 506a configured to receive and seat one or more drive cables, such as the third and fourth drive cables 408c,d of FIG. 4, to effect such movement (rotation). The second blade holder 502b is configured to receive and seat the second blade 212 such that movement (rotation) of the second blade holder 502b about the first pivot axis P₁ correspondingly moves (rotates) the second blade 212. The second blade holder 502b may also provide and otherwise define a pair of second tracks or pulleys 504b and 506b configured to receive and seat one or more drive cables, such as the first and second drive cables 408a,b of FIG. 4, to effect such movement (rotation).

As used herein, the term “blade holder” is intended to apply to a variety of types of end effectors having opposing blades or jaws that are movable relative to one another. In the illustrated embodiment, the blades 210, 212 comprise opposing scissor blades of a surgical scissors end effector. In other embodiments, however, the blades 210, 212 may alternatively comprise opposing jaws used in a grasper end effector, or the like, and the term “jaw holder” similarly applies, without departing from the scope of the disclosure. Moreover, the term “holder” in “blade holder” may be replaced with “mount,” “drive member,” or “actuation member.”

In some embodiments, the first and second blade holders 502a,b may be made of an electrically insulating or non-conductive material. Suitable non-conductive materials include, but are not limited to, a ceramic (e.g., zirconia, alumina, aluminum nitride, a silicate, silicon nitride, etc.), high temperature and high strength plastics, a thermoplastic or thermosetting polymer (e.g., polyether ether ketone, ULTEM™, VESPEL®, a polyphenylsulfone, a polysulfone, RADEL®, a polyamide-imide, a polyimide, an epoxy, etc.), a composite material (e.g., fiberglass), hard rubber (e.g., ebonite), or any combination thereof. Alternatively, the proximal region of the blades 210, 212 may be coated in a nonconductive material (e.g., ceramic) to isolate the proximal regions of the blades 210, 212 from the blade holders 502a,b that isolated the blades 210, 212 from the rest of the wrist components, thus allowing these wrist components to be constructed out of a traditional conductive material such as stainless steel.

In some embodiments, the first and second blade holders 502a,b may each comprise a monolithic structure made of a common (singular) material. In other embodiments, however, one or both of the blade holders 502a,b may comprise two or more portions joined together to form the blade holder 502a,b. In such embodiments, for example, a first portion of the blade holder 502a,b may be configured to receive the blade 210, 212 and the second portion may provide the corresponding pulley 504a,b. Moreover, in such embodiments, the first and second portions may be made of the same or dissimilar materials. The first portion, for example, may be made of a non-conductive material (e.g., ceramic or a polymer) and the second portion may be made of a dissimilar non-conductive material or alternatively a conductive material. In other embodiments, the first and second portions may be made of dissimilar non-conductive materials. In such embodiments, the first portion may be made of ceramic, and the second portion may be made of a plastic over-molded onto the first portion and otherwise coupled thereto.

The first and second blades 210 and 212 may comprise or otherwise form part of a removable blade assembly 500 (hereinafter, “the blade assembly 500”). The first and second blade holders 502a,b are configured to removably receive the blade assembly 500, such that the first and second blades 210 and 212 may be quickly removed from the end effector 204 and then replaced as desired. Thus, the blade assembly 500 may be inserted into the end effector 204 in preparation for performing a surgical procedure, and the blade assembly 500 may subsequently be removed and either sent to cleaning/sterilization or discarded after completion of the surgical procedure.

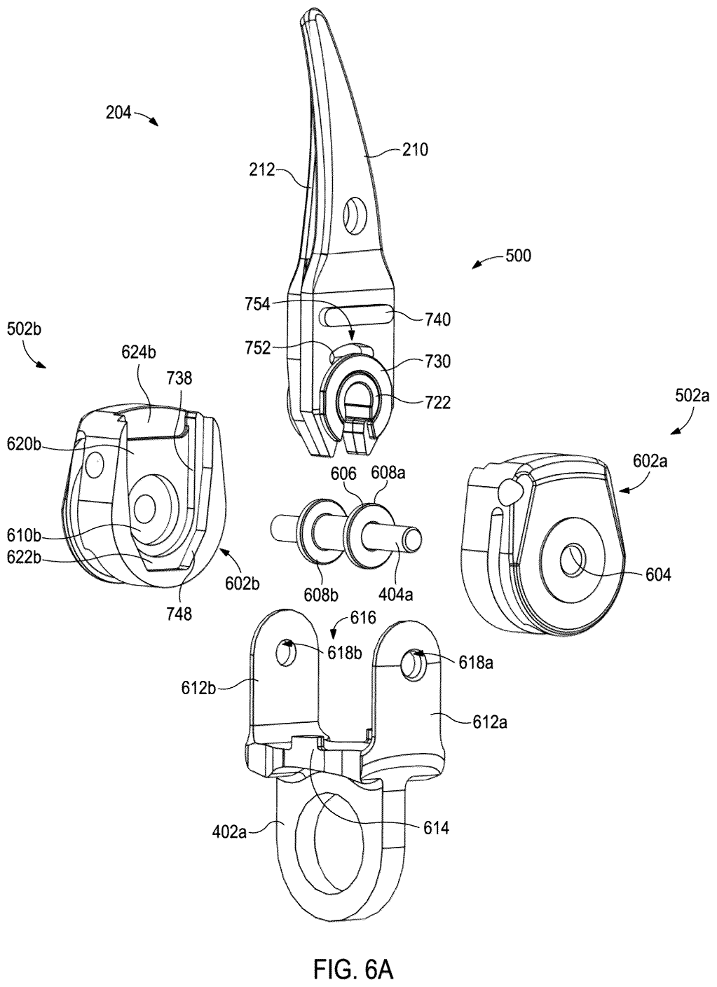

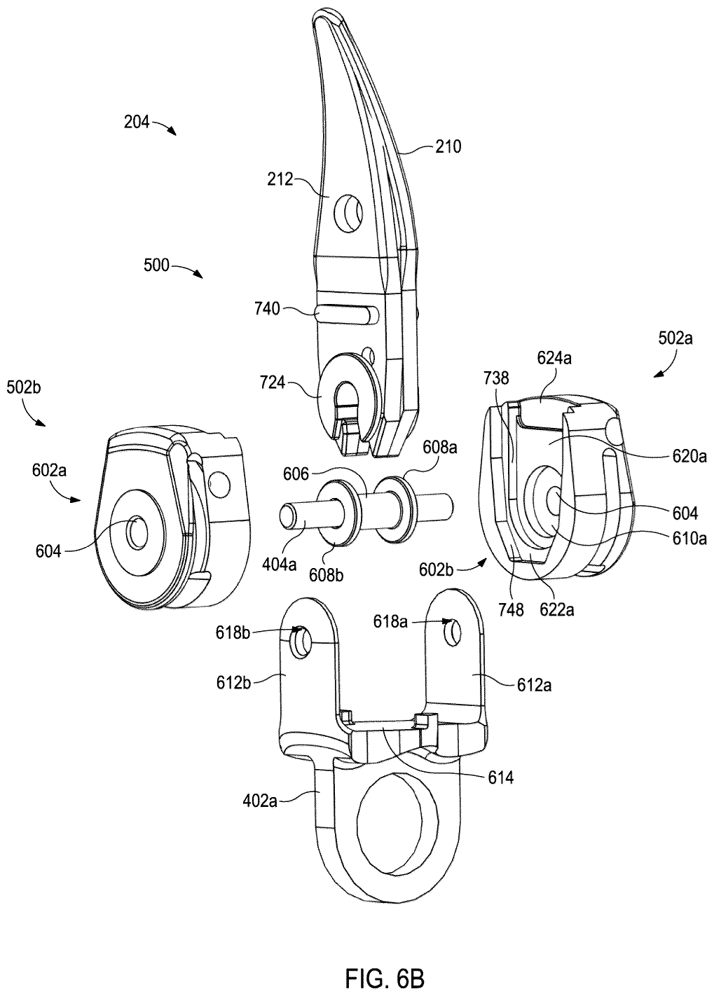

FIGS. 6A and 6B are exploded front-left and rear-right isometric views, respectively, of the end effector 204 of FIGS. 5A-5C, according to one or more embodiments. More specifically, FIGS. 6A-6B show the blades 210, 212 and corresponding blade holders 502a,b removed and exploded away from the first axle 404a, and the first axle 404a exploded away from the distal clevis 402a.

Each blade holder 502a,b includes an outer side 602a and an inner side 602b that is opposite the outer side 602a. As illustrated, an axle aperture 604 is defined in each of the blade holders 502a,b for receiving the first axle 404a. The axle aperture 604 extends fully through each of the blade holders 502a,b, between the outer and inner sides 602a,b. Also, a bushing 606 is arranged around (mounted to) the first axle 404a. As briefly mentioned above, the blades 210, 212 are part of the blade assembly 500 that is installable within the blade holders 502a,b for use in a surgical procedure, and then removed from the blade holders 502a,b and disposed of following such use. As further described below, the blade assembly 500 is configured to be removably mounted to the bushing 604, thereby removably mounting the blades 210, 212 to the first axle 404a.

A pair of flanged ends 608a,b are defined on and otherwise form part of the bushing 606. The flanged ends 608a,b are enlarged radial shoulders provided at opposite sides (ends) of the bushing 606, and the inner sides 602b of the blade holders 502a,b are configured to receive or accommodate the flanged ends 608a,b. In the illustrated embodiment, a first recess 610a is formed in the inner side 602b of the first blade holder 502a and is sized to receive the first flanged end 608a of the bushing 606 when assembled, and a first recess 610b is formed in the inner side 602b of the second blade holder 502b and is sized to receive the second flanged end 608b of the bushing 606 when assembled. The bushing 606 may be made of various types of materials, such as a high strength material or nylon. In some embodiments, the bushing 606 is made from stainless steel or a corrosion resistant alloy. Also, in some embodiments, and in order to increase the wear resistance of the axle 404a, the outer surface of the axle 404a may be coated and otherwise include a coating on an outer surface thereof. For example, a diamond-like Carbon (“DLC”) coating may be applied to the outer surface of the axle 404a.

As shown, the distal clevis 402a provides opposing first and second arms 612a and 612b laterally offset from each other and extending distally from a main body 614. A space or gap 616 is formed between the arms 612a,b and is sized to receive the blade holders 502a,b as mounted to the first axle 404a, with the bushing 606 arranged on the first axle 404a and between the inner sides 602b of the blade holders 502a,b when assembled. Each arm 612a,b provides and otherwise defines an opening 618a and 618b, respectively, and the openings 618a,b are aligned with each other and configured to receive and seat opposing ends of the first axle 404a. Accordingly, the blade holders 502a,b are rotatably supported by and between the arms 612a,b such that the blade holders 502a,b are rotatable on the first axle 404a about the second pivot axis P₂ and relative to the distal clevis 402a. Consequently, the blade assembly 500 will be similarly rotatable about the second pivot axis P₂ when installed within the blade holders 502a,b, as detailed herein.

The inner side 602b of each blade holder 502a,b is configured to receive and seat the corresponding blade 210, 212 such that movement (rotation) of the blade holder 502a,b will correspondingly move (rotate) the corresponding blade 210, 212. As illustrated, in addition to the first recess 610a, the inner side 602b of the first blade holder 502a also defines a second recess 620a and a third recess 622a. Here, the first recess 610a is formed within the first blade holder 502a such that it extends into the inner side 602b thereof (towards the outer side 602a). The second recess 620a and the third recess 622a are also formed within the inner side 602b of the first blade holder 502a, such that they also extend therein towards the outer side 602a, but to a lesser extent than the first recess 610a. For example, the first recess 610a extends deeper into the inner side 602a than the second recess 620a, and the second recess 620a extends deeper into the inner side 602b than the third recess 622a.

Also, in addition to the first recess 610b defined in the second blade holder 502b, the inner side 602b of the second blade holder 502b also defines a second recess 620b and a third recess 622b. Similar to the first blade holder 502a, first recess 610b extends deeper into the inner side 602a of the second blade holder 502b than the second recess 620b, and the second recess 620b extends deeper into the inner side 602b of the second blade holder 502b than the third recess 622b.

The third recess 622a,b of the blade holders 502a,b are each configured to receive and seat the corresponding blade 210, 212 such that movement (rotation) of the blade holder 502a,b will correspondingly move (rotate) the corresponding blade 210, 212. Each second recess 620a,b extends deeper into the material of the blade holder 502a,b than the third recess 622a,b and may be configured to receive a retainer of the blade assembly, as detailed below. Lastly, as mentioned above, the first recesses 610a,b are configured to receive the flanged end 608a,b of the bushing 606.

In addition, the inner side 602b of each of the blade holders 502a,b includes and otherwise defines a pocket or slot 624a,b configured to engage (receive) a portion (i.e., a boss) of the associated blade 210, 212, as will be discussed below. This will help ensure that the blades 210, 212 of the blade assembly 500 are fully seated and installed on the first axle 404a and within the blade holders 502a,b, and will provide a means for the user to visually confirm such proper seating and installation.

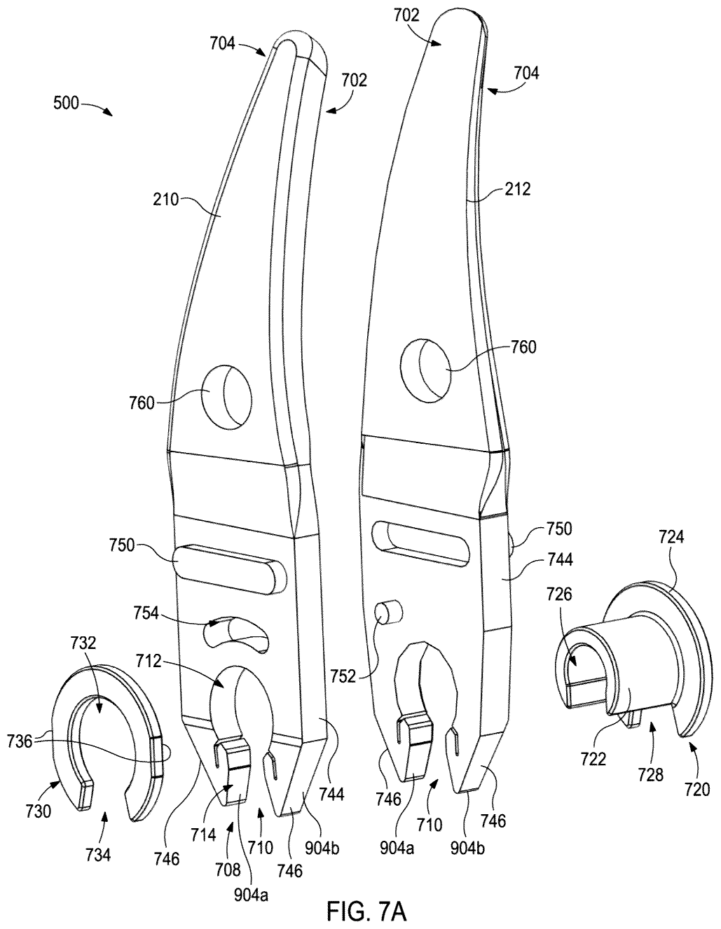

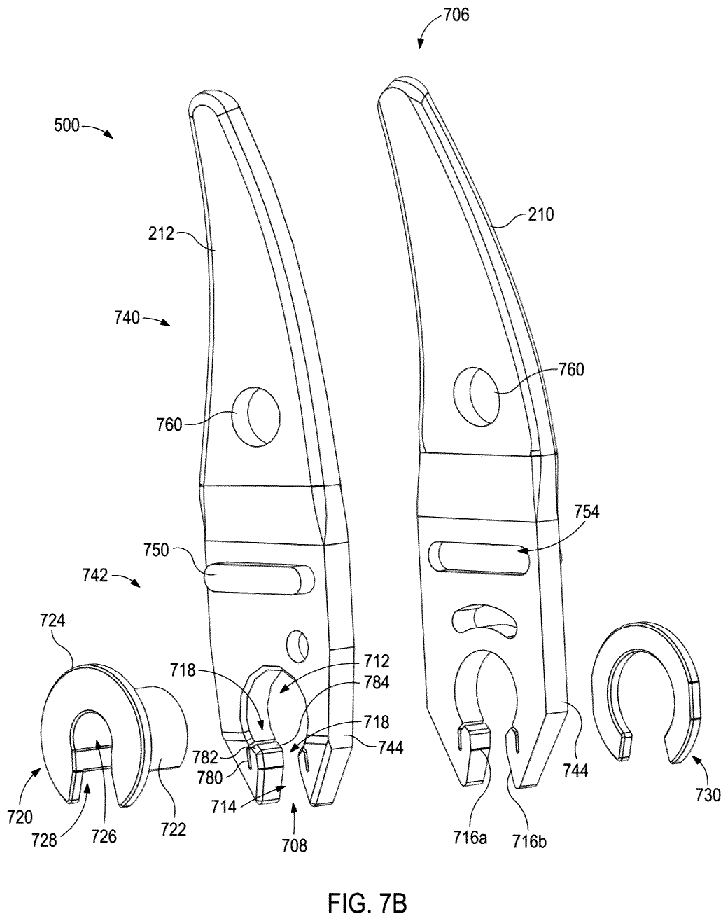

FIGS. 7A and 7B are exploded front-right and rear-left isometric views, respectively, of the blade assembly 500 of the end effector 204 of FIGS. 5A-5C, according to one or more embodiments. Each of the blades 210, 212 includes an inner side 702 and an opposing outer side 704, and opposing distal and proximal ends 706, 708. Each blade 210, 212 also includes an open-ended axle slot 710 that opens in the proximal direction (i.e., each of the open-ended axle slots 710 has an opening at the proximal end 708). The blades 210, 212 are configured to be removably coupled to the first axle 404a (FIGS. 6A-6B) at the open-ended axle slots 710.

Each open-ended axle slot 710 includes an axle opening 712 and an axle slot 714 that extends to and is contiguous with the axle opening 712. When assembled, the first axle 404a is received within the axle opening 712 and the axle slot 714 is configured to permit the blade assembly 500 to be snap fit onto the first axle 404a and then removed therefrom. The axle slot 714 may be defined by a pair of opposing surfaces 716a,b that are oriented/slanted away from each other towards the proximal end 708 of the axle slot 714. As a result, a gap is defined between the opposing surfaces 716a,b and expands towards the proximal end 708 (i.e., the gap is the largest at the end of the axle slot 714 opposite the axle opening 712). The gap diminishes in size towards the axle opening 712 and is the smallest (or narrowest) at a constricted portion 718 (FIG. 7B) of the open-ended axle slot 710, wherein the constricted portion 718 is positioned in between the axle opening 412 and the axle slot 714. The constricted portion 718 helps maintain and retain the first axle 404a within the axle opening 712 until the user pulls/withdraws the blade assembly 700 in a distal direction with sufficient force to overcome the natural bias of the proximal ends 708 of the blades 210,212 and thereby allow the blade assembly 700 to be dislodged from the axle 404a and then withdrawn from over the first axle 404a.

The blade assembly 500 also includes a retainer 720 for coupling the blades 210, 212 together. The blades 210, 212 are assembled on the retainer 720 such that the inner sides 702 of the blades 210, 212 face each other. The retainer 720 extends through the open-ended axle slots 710 of each blade 210, 212. In particular, the retainer 720 is arranged in (extends into) the axle opening 712 of each open-ended axle slot 710, such that the blades 210, 212 may each rotate over and relative to the retainer 720, and such that the blades 210, 212 may rotate relative to each other as well.

The retainer 720 includes a generally cylindrical shaped body 722 and an enlarged radial shoulder 724 defined on and otherwise forming part of the body 722 at a first end of the body 722. In the illustrated embodiment, an inside surface 725 (FIG. 7A) of the shoulder 724 will contact or abut the outer side 704 of the second blade 212 when the retainer 720 is assembled on the blades 210, 212. In other embodiments, however, the shoulder 724 may contact or abut the outer side 704 of the first blade 210 when the retainer 720 is assembled on the blades 210, 212. Also, when the blade assembly 500 is installed within the blade holders 502a,b, the shoulder 724 will be received within the second recess 620a (FIGS. 6A-6B) defined in the inner side 602b of the first blade holder 502a. Accordingly, the shoulder 724 is dimensioned/sized and shaped to be slidingly received within the profile of the second recess 620a and/or vice versa. However, as mentioned below, the blade assembly 500 may be inserted into the blade holders 502a,b with the shoulder 724 being received in the second recess 620b in the second blade holder 502b, such that the second recesses 620a,b may be similarly dimensioned to receive the shoulder 724.

When the blade assembly 500 is installed within the end effector 204, the retainer 720 will be arranged over and on the first axle 404a. Thus, a bore 726 is defined within the retainer 720 and extends through the body 722, between the opposing axial ends of the retainer 720. Further, and to allow the blade assembly 500 to be snap fit onto the first axle 404a and thereafter removed, the retainer 720 includes an opening 728 that extends through the body 722 and the shoulder 724 and into the bore 726. Thus, the opening 728 of the retainer 720 is in communication with (contiguous with) the bore 726 and, due to the presence of the bore 726 and the opening 728 in the retainer 720, the body 722 of the retainer 720 resembles a C-shaped body.

When the retainer 720 is assembled on the blades 210, 210, the opening 728 of the retainer 720 may be generally aligned with the open-ended axle slots 710 of the blades 210, 212. Thus, when the user inserts the blade assembly 500 into the blade holders 502a,b, the blades 210, 212 may be received on the first axle 404a and, more particularly, on the bushing 606 mounted to the first axle 404. The bushing 606 may be sized to be received within the open-ended axle slots 710, which flex laterally outward to accommodate the bushing 606. The retainer 720 is received within the axle opening 712, which allows the bushing 606 to pass through the opening 728 to be received within the bore 726 of the retainer 720.

Further, as shown, the blade assembly 500 includes a spacer 730. When assembled, the spacer 730 is arranged at the second end of the retainer 720 opposite the shoulder 724. The spacer 730 includes a bore 732 (FIG. 7A) sized and dimensioned to receive (extend over) the second end of the body 722 of the retainer 720. Also, the spacer 730 includes an opening 734 (FIG. 7A) that extends into and is contiguous with the bore 732 such that the spacer 730 resembles a C-shaped member.

When the spacer 730 is assembled on the blade assembly 500, the opening 734 is generally aligned with the opening 728 of the retainer 720. In some embodiments, the opening 734 of the spacer 730 may be sized slightly larger and with similar geometry to the opening 728 at the second end of the body 724, such that the retainer 720 may be pressed onto the first axle 404a without the spacer 730 impeding movement of the retainer 720 or otherwise interfering with the first axle 404a (or the bushing 606) as the first axle 404a (e.g., the bushing 606) is received within the retainer 720.

In the illustrated embodiment, the spacer 730 will contact or abut the outer side 704 of the first blade 210 when assembled on the retainer 720. However, in other embodiments, the spacer 730 may contact or abut the outer side 704 of the second blade 212 when the spacer 730 is assembled on the retainer 720. Also, when the blade assembly 500 is installed within the blade holders 502a,b, the spacer 730 will be arranged within the second recess 620b (FIGS. 6A-6B) defined in the inner side 602b of the second blade holder 502b. Accordingly, the spacer 730 is dimensioned/sized and shaped to be slidingly received within the second recess 620b and/or vice versa.

In the illustrated embodiment, the spacer 730 is keyed to the second recess 620b to inhibit relative rotation between the spacer 730 and the second blade holder 502b. More specifically, in some embodiments, a portion of the periphery of the spacer 730 may include flat surfaces 736 (FIG. 7A) on either side of the spacer 730, and the flat surfaces 736 may be configured to abut corresponding flat surfaces 738 (see FIG. 6B) in the periphery of the second recess 620a (FIGS. 6A-6B) that is defined in the inner side 602b (FIGS. 6A-6B) of the first blade holder 502a. In the illustrated embodiment, the flat surfaces 738 are also formed in the periphery of the second recess 620b of the second blade holder 502b (see FIG. 6A), such that the blade assembly 500 may be inserted into the blade holders 502a,b in either an orientation as depicted in the figures (i.e., with the first blade 210 and the spacer 730 in the first blade holder 502a, and with the shoulder 724 of the retainer 720 and the second blade 212 in the second blade holder 502b) or in an opposite orientation (i.e., with the first blade 210 and the spacer 730 in the second blade holder 502b, and with the shoulder 724 of the retainer 720 and the second blade 212 in the first blade holder 502a). Thus, the second recesses 620a,b may each include the flat surfaces 738 keyed to the spacer 730, and may also be configured to receive the generally circular shoulder 724 of the retainer 720.

Still referring to FIGS. 7A and 7B, each of the blades 210, 212 may be keyed to their respective blade holder 502a,b such that the blades 210, 212 rotate with their respective blade holder 502a,b. Stated differently, each of the blades 210, 212 may be keyed to their respective blade holder 502a,b such that the rotation of the blade holders 502a,b correspondingly imparts rotation to the respective blade 210, 212. As shown, the blades 210, 212 each include a blade portion 740 and a base portion 742, wherein the blade portion 740 includes a cutting edge that is operable to cut and/or manipulate tissue, and the base portion 742 is proximal from the blade portion 740 and extending distally from the blade portion 742. An outer periphery 744 of the base portion 742 may be keyed to the respective blade holder 502a,b, such that the first and second blade 210, 212 move with and are carried by the first and second blade holders 502a,b, respectively. More specifically, the third recesses 622a,b of the first and second blade holder 502a,b, respectively, are sized and shaped to receive and retain the outer periphery 744 of the first and second blades 210, 212, respectively.

Keying the blades 210, 212 to the respective blade holder 502a,b in this manner helps to transfer movement (rotation) from the blade holders 502a,b to the corresponding blade 210, 212. Thus, when the blade assembly 500 is fully installed and seated within the blade holders 502a,b, the periphery 744 of the blades 210, 212 will contact the periphery of the corresponding third recesses 622a,b such that rotation of the blade holders 502a,b will correspondingly rotate the blade 210, 212 received therein. Accordingly, the outer periphery 744 of the first blade 210 is keyed to the recess 622a of the first blade holder 502a such that the first blade holder 502a carries the first blade 210 as the first blade holder 502a rotates about the first pivot axis P₁, and the outer periphery 744 of the second blade 212 is keyed to the recess 622b of the second blade holder 502b such that the second blade holder 502b carries the second blade 212 as the second blade holder 502b rotates about the first pivot axis P₁.

In the illustrated embodiment, the outer periphery 744 of each blade 210, 212 includes a pair of chamfered or beveled sides 746 (FIG. 7A) formed at the proximal end 708 of the base portion 742. In the illustrated embodiment, the beveled sides 746 are slanted/sloped towards each other when evaluated from the distal end 706 towards the proximal end 708. Thus, a width of the blades 210, 212 diminishes when evaluated towards the proximal end 708. The presence of the chamfered or beveled sides 746 may facilitate insertion of the blades 210, 212 (when coupled together in the form of the blade assembly 500) into the recesses 622a,b defined in the blade holders 502a,b, by inhibiting any portion of the blades 210, 212 from being caught on a portion of the blade holder 502a,b that would otherwise hinder proximal insertion of the blade assembly 500 within the end effector 204. When the blades 210, 212 are fully seated within their corresponding blade holder 502a,b, the chamfered or beveled sides 746 will abut/contact a corresponding side 748 (see FIGS. 6A and 6B) of the recesses 622a,b defined in each of the blade holders 502a,b.

In the illustrated embodiment, each of the blades 210, 212 also includes a blade holder interface or “boss” 750 protruding or extending from the outer side 704. Each boss 750 is sized and shaped to fit within the corresponding slot 624a,b (FIGS. 6A-6B) of the associated blade holder 502a,b. For example, the slot 624a of the first blade holder 502a receives the boss 750 of the first blade 210 when the blade assembly 500 is installed, and the slot 624b of the second blade holder 502a receives the boss 750 of the second blade 212 when the blade assembly 500 is installed. Receiving the bosses 750 in the slots 624a,b helps key the blades 210, 212 to the blade holders 502a,b during rotation (operation), and may also prove advantageous in providing a means of ensuring that the blades 210, 212 are fully seated and installed on the first axle 404a and within the blade holders 502a,b.

As illustrated, the second blade 212 includes a projection 752 (FIG. 7A) configured to be received within an arcuate slot 754 defined in the first blade 211 when the blades 210, 212 are coupled together via the retainer 720. The arcuate slot 754 extends through the first blade 210 between the inner and outer sides 702, 704. Receiving the projection 752 in the arcuate slot 754 helps prevent the blades 210, 212 from over-rotating (in both angular directions) during operation. The arcuate slot 754 has an arc length that limits (defines) the amount of travel that the projection 752 may undertake as the blades 210, 212 pivot relative to one another. It should be appreciated that while the projection 752 and the arcuate slot 754 are illustrated on the second blade 212 and the first blade 210, respectively, in other embodiments, the arcuate slot 754 may be formed in the second blade 212 and the projection 752 may be provided on the first blade 210.

In addition, the blade assembly 500 may be configured to facilitate removal of the blade assembly 500 from the end effector 204. In the illustrated embodiment, each of the blades 210, 212 includes an aperture 760 used to facilitate removal of the blade assembly 500 from the end effector 204. The apertures 760 are provided on the blade portion 740 of each of the blades 210, 212, but could alternatively be provided elsewhere and accessible by the user when the blade assembly 500 is installed in the end effector 204. In some embodiments, a tool may be utilized to access (engage) the apertures 760 and thereby withdraw the blade assembly 500. For example, the blade assembly 500 may be pre-sterilized and packaged in a cartridge or container (not illustrated). In this example, the surgical tool 200 (without the blade assembly 500) may be delivered to the operating room for use, at which time the blade assembly 500 may be installed within the end effector 204 of the surgical tool 200 using the cartridge or container to ensure that the blade assembly 500 is maintained in a sterile condition when installed. After use, the cartridge or container that previously housed the sterile blade assembly 500 may be used to remove the blade assembly 500 from the end effector 204.

Also in the illustrated embodiment, each of the blades 210, 212 includes a pair of proximal prongs 904a,b and a cut or slot 780 is formed in each of the proximal prongs 904a,b. The cut or slot 780 extends into the open-ended axle slot 710 and, more particularly, opens into the axle opening 712 of the open-ended axle slot 710. As shown, the presence of the cut or slot 780 on each of the proximal prongs 904a,b defines a respective barb 782 on each of the proximal prongs 904a,b that will exhibit a natural bias when urged/pressed away from its naturally extending position. Here, each of the barbs 782 also includes an angled or chamfered surface 784 that will aid in withdrawing or uninstalling the blade assembly 500 from the end effector 204.

For example, when attempting to withdraw the blade assembly 500 from the end effector 204, the first axle 404a and the bushing 606 will contact the angled or chamfered surface 784 of the barbs 782 and, as the first axle 404a and the bushing 606 slide on the angled or chamfered surface 784 they will impart a force on the barbs 782 and urge the barbs 782 outward or away from the constricted portion 718 of the open-ended axle slot 710, thereby allowing the blade assembly 500 to be pulled off of the first axle 404a and the bushing 606. The barbs 782 may also help with retaining the blade assembly 500 on the bushing 606 and the first axle 404a by providing a snap-fit interface, wherein the barbs 782 deflect outward as the blade assembly 500 travels over the bushing 606 and the first axle 404a and then the barbs 782 deflect back into their natural (undeformed) position due to the natural bias of the material utilized to construct the blades 210, 212, thereby operating to trap the bushing 606 and the first axle 404a within the axle opening 712 of the open-ended axle slot 710.

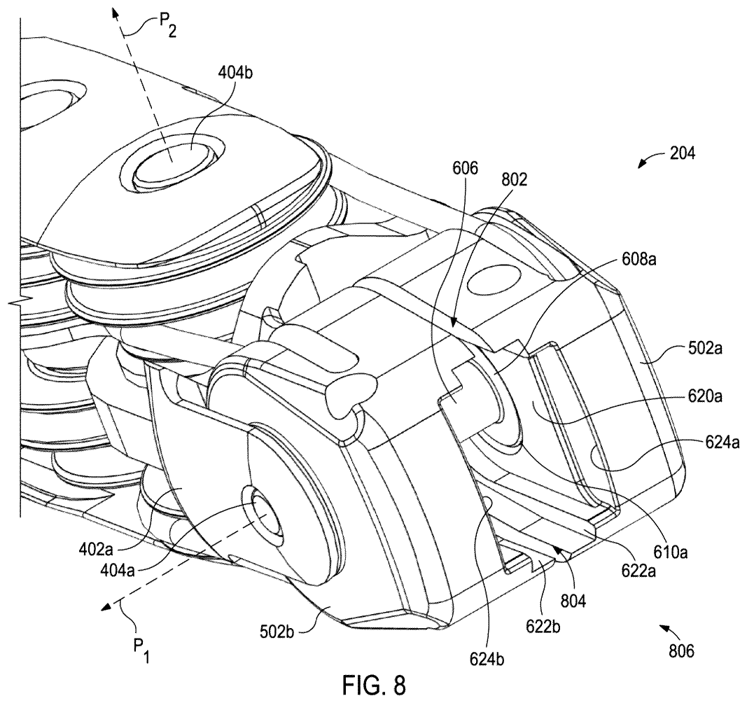

FIG. 8 depicts the distal end of the end effector 204 without the blade assembly 500 (FIGS. 7A-7B) inserted therein, according to one or more embodiments of the disclosure. As illustrated, when the blade holders 502a,b are assembled on the first axle 404a, the bushing 606 is arranged on the first axle 404a and located between the blade holders 502a,b with the flanged ends 608a,b of the bushing 608 disposed within the first recesses 610a,b, and gap 802 is defined between the blade holders 502a,b. The gap 802 provides a space between the blade holders 502a,b such that they do not rub/abrade on each other during relative rotation.

As shown, the blade holders 502a,b cooperatively define an insertion slot 804 at a distal end 806 of the end effector 204 and configured to receive and allow the blade assembly 500 to be snap-fit onto the bushing 606. In some embodiments, as illustrated, the insertion slot 804 is defined between the recesses 620a,b and 622a,b, and the slots 624a,b.

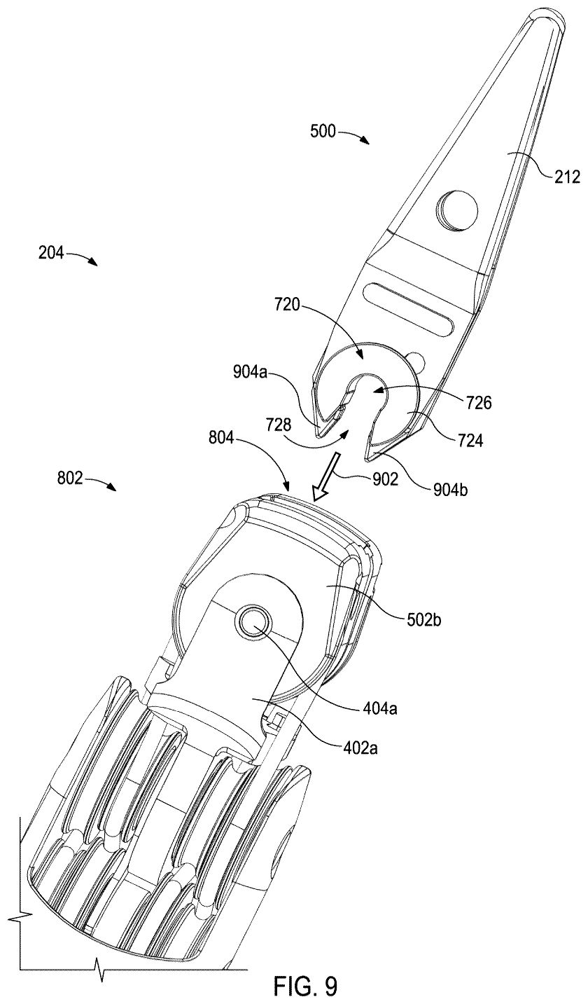

FIG. 9 depicts example assembly of the end effector 204, according to one or more embodiments. More specifically, FIG. 9 depicts example installation of the blade assembly 500 within blade holders 502a,b. To install the blade assembly 500, a user advances the blade assembly 500 toward the end effector 204 such that the proximal end 708 of the blades 210, 212 are received within the insertion slot 804, as indicated by arrow 902. The proximal end 708 of the blades 210, 212 pass over the bushing 606 (FIG. 8), which is received between the opposing surfaces 716a,b (FIGS. 7A-7B) of the blades 210, 212. The slanted/sloped orientation of the opposing surfaces 716a,b causes proximal prongs 904a,b of the blades 210, 212 to deflect or flex laterally outward such that the blades 210, 212 can travel over the bushing 606. As the blade assembly 500 is advanced further into the insertion slot 804 in the proximal direction 902, the proximal prongs 904a,b pass beyond the bushing 606 and the opening 728 in the retainer 720 will receive the bushing 606.

Further movement of the blade assembly 500 in the proximal direction 902 will result in the bushing 606 being positioned/seated within the bore 726 of the retainer 720, at which time the proximal prongs 904a,b of the blades 210, 212 (as well as the retainer 720) are able to deflect (or snap) back into their natural or undeflected position (state), such that the blade assembly 500 is snap-fit in place.

After using the end effector 204, the blade assembly 500 may be removed from the blade holders 502a,b by reversing the forgoing process.

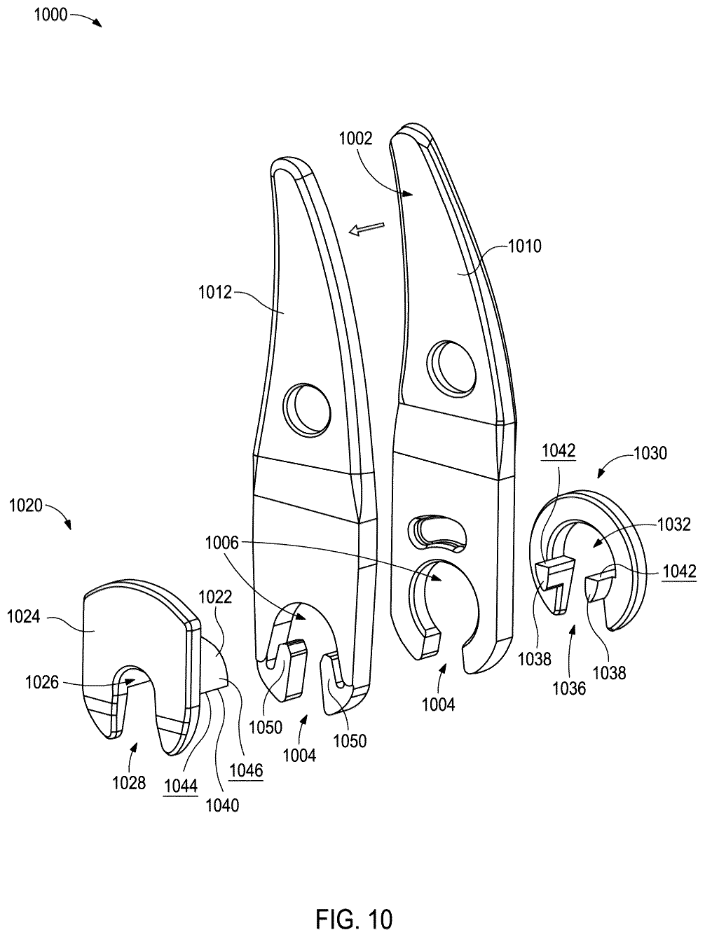

FIG. 10 depicts an alternate removable blade assembly 1000, according to one or more embodiments of the present disclosure. As with the blade assembly 500, the removable blade assembly 1000 (hereinafter, “the blade assembly 1000”) includes first and second blades 1010, 1012, and the first and second blade holders 502a,b (FIGS. 5A-5B) are configured to removably receive the blade assembly 1000, such that the first and second blades 1010 and 1012 may be quickly removed from the end effector 204 and then replaced as desired. Thus, the blade assembly 1000 may be inserted into the end effector 204 in preparation for performing a surgical procedure, and the blade assembly 1000 may subsequently be removed and either sent to cleaning/sterilization or discarded after completion of the surgical procedure.

As illustrated, the blade assembly 1000 includes a retainer 1020 and a spacer 1030 for coupling the blades 1010, 1012 together. The blades 1010, 1012 are assembled on the retainer 1020 such that inner sides or surfaces 1002 of the blades 1010, 1012 face each other. The retainer 1020 includes a body 1022 and an enlarged shoulder 1024 defined on and otherwise forming part of the body 1022 at a first end of the body 1022. A bore 1026 extends through the body 1022 and the shoulder 1024, and the bore 1026 provides an opening 1028 that extends into and is contiguous with the bore 1026. In the illustrated embodiment, an inside surface of the shoulder 1024 will contact or abut an outer side or surface of the second blade 1012 when the retainer 1020 is assembled on the blades 1010, 1012. In other embodiments, however, the shoulder 1024 may contact or abut an outer surface of the first blade 1010 when the retainer 1020 is assembled on the blades 1010, 1012.

Each blade 1010, 1012 includes an open-ended axle slot 1004, and the body 1022 of the retainer 1020 is sized to extend through the co-axially aligned open-ended axle slots 1004. The open-ended axle slots 1004 are also sized to be large enough such that they may receive (and traverse) the bushing 606 and the first axle 404a (FIGS. 6A-6B) as detailed herein. In particular, the body 1022 of the retainer 1020 is configured to be arranged in (extend into and through) an axle opening portion 1006 of each open-ended axle slot 1004, such that the blades 1010, 1012 may each rotate over and relative to the retainer 1020, and such that the blades 1010, 1012 may rotate relative to each other as well. Thus, the axle opening portion 1006 of each open-ended axle slot 1004 is sized and dimensioned to receive and fit on the body 1022 of the retainer 1020.

The spacer 1030 includes a bore 1032 sized and dimensioned to receive (extend over) the end of the body 1022 of the retainer 1020 opposite the shoulder 1024. The spacer 1030 further includes an opening 1036 that extends into and is contiguous with the bore 1032 such that the spacer 1030 resembles a C-shaped member. When assembled, the spacer 1030 is arranged with the body 1022 of the retainer 1020 received in the bore 1032 of the spacer 1030, and with the opening 1036 of the spacer 1030 being aligned with the opening 1028 in the retainer 1020. To facilitate proper alignment between the retainer 1020 and the spacer 1030, the spacer 1030 includes a pair of projections or prongs 1038 that will abut and contact a corresponding edge 1040 of the body 1022 of the retainer 1020. In particular, when the body 1022 of the retainer 1020 is advanced into the bore 1032 of the retainer 1030, upward facing surfaces 1042 of the prongs 1038 will abut/contact corresponding downward facing surfaces 1044 at the edges 1040 of the body 1022 of the retainer 1020.

When assembled on the end effector 204, the bushing 606 (FIGS. 6A-6B) (on the first axle 404a) will be received in the bore 1026 of the retainer 1020. Thus, the openings 1028, 1036 in the retainer 1020 and the spacer 1030, respectively, will be co-axially aligned when assembled, and are sized and dimensioned to move over and receive the bushing 606 when installing or removing the blade assembly 1000 from the end effector 204. Also, portions of the blades 1010, 1012 within their axle opening portions 1006 will contact/abut a bearing surface 1046 of the body 1022 of the retainer 1020, and the bearing surface 1046 will function as a journal surface for the blade assembly 1000 when fully assembled and installed in the end effector 204.

The second blade 1012 may further include snap features 1050. The snap features 1050 may flex, providing the snap features 1050 with a spring functionality. The snap features 1050 are designed to snap over the bushing 606 arranged on the first axle 404a. When the blade assembly 1000 is fully assembled and installed in the end effector 204, the snap features 1050 help retain the blade assembly 1000 within the end effector 204, as removal of the blade assembly 1000 from the end effector 204 would require pulling the blade assembly 1000 out of the end effector 204 with sufficient withdrawal force/load that overcomes the force applied by the natural bias of the snap features 1050. Stated differently, to remove the blade assembly 1000, it would need to be pulled with sufficient force/load to cause the snap features 1050 to flex outward (away from the open-ended axle slot 1004) and thereby allow the second blade 1012 to be pulled over the bushing 606. The snap features 1050 may be designed with a small amount of interference when snapping onto or off the bushing 606 to allow installation/removal of the blade assembly 100 with a safe and amount installation and removal loads as may be desired to install or remove the blade assembly 1000.

In embodiments, the various components of the blade assembly 1000 are separable from each other and retained together by coupling the retainer 1020 and the spacer 1030, which maintain the blades 1010, 1012 together in a preloaded state. In other embodiments, one or more of the components of the blade assembly 1000 may be attached to each other, for example, via welding. For example, the retainer 1020, the spacer 1030 and the second blade 1012 (with the snap features 1050) may be welded together, thereby trapping the first blade 1010 between the second blade 1012 and the spacer 1030, and thereby allowing rotational movement of the first blade 1010 on/around the body 1022 of the retainer 1020 relative to the second blade 1012. By attaching the second blade 1012, the retainer 1020, and the spacer 1030 all together as an integral unit, it will make it easier to align the second blade 1012, the retainer 1020, and the spacer 1030, such that the opening 1028 in the retainer 1020 and the opening 1036 in the spacer 1030 are aligned with each other and with the open-ended axle slot 1004 of the second blade 1012. Moreover, such alignment will facilitate installation and removal of the blade assembly 1000. To facilitate removal and/or installation, the first blade 1010 may be aligned with the second blade 1012 such that their open-ended axle slot 1004 are aligned before installing or removing.

In these embodiments, in order to rotate the second blade 1012, the second blade holder 502b (FIGS. 5A-5B) could impart rotational movement on either or both the retainer 1022 and the second blade 1012 to cause rotation of the second blade 1012, as the second blade 1012 and the retainer 1020 are welded together. As to the first blade 1010, the first blade holder 502a (FIGS. 5A-5B) could impart rotational movement directly on the first blade 1010 to cause rotation of the first blade 1010 (relative to the second blade 1012, the retainer 1020, and the spacer 1030). Thus, the blade holders 502a,b are operable to translate motion from the cables 408a-d to the blades 1010, 1012.

Also, when the blade assembly 500 is installed within the blade holders 502a,b, the shoulder 724 will be received within the second recess 620a (FIGS. 6A-6B) defined in the inner side 602b of the first blade holder 502a. Accordingly, the shoulder 724 is dimensioned/sized and shaped to be slidingly received within the profile of the second recess 620a and/or vice versa. However, as mentioned below, the blade assembly 500 may be inserted into the blade holders 502a,b with the shoulder 724 being received in the second recess 620b in the second blade holder 502b, such that the second recesses 620a,b may be similarly dimensioned to receive the shoulder 724.

Embodiments disclosed herein include:

A. A replaceable blade assembly for an end effector of a surgical tool, including first and second blades, each blade having opposing inner and outer sides, opposing distal and proximal ends, and an open-ended slot provided at the proximal end that provides an axle slot that extends into an axle opening of the open-ended slot, and a retainer extendable through the axially-aligned axle openings and operable to couple the first and second blades together with the inner side of each blade facing each other, while permitting relative rotation between the first and second blades.

B. A surgical tool includes a drive housing, an elongate shaft extending distally from the drive housing, and a wrist arranged at a distal end of the shaft and including a clevis providing a main body and opposing first and second arms extending distally from the main body, an opening defined in each arm, and an axle mounted to the clevis and extending through each opening. The surgical tool further including an end effector operatively coupled to the wrist and including first and second blade holders rotatably mounted to the axle and arranged between the first and second arms, and a blade assembly removably mounted to the axle and including a first blade received at the first blade holder, and a second blade received at the second blade holder, wherein the first and second blades each include an open-ended slot provided at a proximal end of the first and second blades, and wherein the blade assembly is rotatably mounted to the axle at the open-ended slot.

C. A method of installing a blade assembly within an end effector of a surgical tool, the method including advancing a proximal end of the blade assembly toward the end effector, the end effector including first and second blade holders rotatably mounted to an axle mounted to a clevis of a wrist of the surgical tool, inserting the proximal end of the blade assembly into an insertion slot cooperatively defined by the first and second blade holders, and removably mounting the blade assembly on the axle within the insertion slot, the blade assembly including a first blade engageable with the first blade holder, and a second blade engageable with the second blade holder, wherein the first and second blades each include an open-ended slot provided at the proximal end, and wherein the removable blade assembly is rotatably mounted to the axle at the open-ended slot.

Each of embodiments A, B, and C may have one or more of the following additional elements in any combination: Element 1: wherein the first and second blades each further include a boss extending from the outer side, and first and second blade holders of the end effector each include an engagement slot for receiving the respective boss when assembled. Element 2: wherein bosses are keyed to their respective engagement slot. Element 3: wherein the first blade includes a projection extending from the inner side thereof, and the second blade includes an arcuate slot formed in the inner side thereof for receiving the protrusion of the first blade, and wherein relative rotation between the first and second blades is limited by an arc length of the arcuate slot. Element 4: wherein the retainer includes a c-shaped body arranged within the axially-aligned axle openings and an enlarged radial shoulder arranged at a first end of the c-shaped body that abuts the outer side of the first blade. Element 5: further comprising a c-shaped spacer arranged at a second end of the c-shaped body that abuts the outer side of the second blade. Element 6: wherein a width of the proximal end of each blade decreases towards the proximal end. Element 7: wherein the open-ended slot includes a constricted portion positioned between the axle slot and the axle opening, and wherein the slot defines a gap that is smallest at the constricted portion.

Element 8: wherein the first and second blades each include opposing inner and outer sides, and the open-ended slot provides an axle slot that extends into an axle opening, the blade assembly further comprising a retainer arranged through axially-aligned axle openings of the open-ended slots and operable to couple the first and second blades together with the inner side of each blade facing each other and while permitting relative rotation between the first and second blades. Element 9: wherein the retainer includes a c-shaped body arranged within the axially-aligned axle openings and an enlarged radial shoulder arranged at a first end of the c-shaped body that abuts the outer side of the first blade. Element 10: further comprising a c-shaped spacer arranged at a second end of the c-shaped body that abuts the outer side of the second blade. Element 11: wherein the first blade and the second blade each include a boss extending from the outer side, and the first and second blade holders each include an engagement slot for receiving the respective boss when assembled, wherein bosses are keyed to their respective engagement slot. Element 12: wherein the first blade includes a projection extending from the inner side thereof, and the second blade includes an arcuate slot formed in the inner side thereof for receiving the protrusion of the first blade, wherein relative rotation between the first blade and the second blade is limited by an arc length of the arcuate slot.

Element 13: further comprising removing the blade assembly from the end effector by dislodging the blade assembly from the axle and withdrawing the blade assembly from the insertion slot. Element 14: further comprising seating a first boss extending from the outer side of the first blade within an engagement slot formed in the first blade holder and seating a second boss extending from the outer side of the second blade within an engagement slot formed in the second blade holder. Element 15: wherein bosses are keyed to their respective engagement slot. Element 16: further comprising limiting relative rotation between the first blade and the second blade via a projection extending from the inner side of the first blade and an arcuate slot formed in the second blade for receiving the protrusion of the first blade, wherein an amount of relative rotation between the first blade and the second blade is defined by an arc length of the arcuate slot. Element 17: wherein a bushing is arranged on the axle and the blade assembly further includes a retainer arranged through axially-aligned axle openings of the open-ended slots for coupling the first and second blades together with the inner side of each blade facing each other and while permitting relative rotation between the first and second, and wherein the method further includes: seating the retainer on the bushing.

By way of non-limiting example, exemplary combinations applicable to A, B, and C include: Element 1 with Element 2; Element 4 with Element 5; Element 8 with Element 9; Element 9 with Element 10; and Element 14 with Element 15.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A replaceable blade assembly for an end effector of a surgical tool, comprising:
first and second blades, each blade having opposing inner and outer sides, opposing distal and proximal ends, and an open-ended slot provided at the proximal end that provides an axle slot that extends into an axle opening of the open-ended slot; and
a retainer extendable through the axially-aligned axle openings and operable to couple the first and second blades together with the inner side of each blade facing each other, while permitting relative rotation between the first and second blades.

2. The replaceable blade assembly of claim 1, wherein the first and second blades each further include a boss extending from the outer side, and first and second blade holders of the end effector each include an engagement slot for receiving the respective boss when assembled.

3. The replaceable blade assembly of claim 2, wherein bosses are keyed to their respective engagement slot.

4. The replaceable blade assembly of claim 1, wherein the first blade includes a projection extending from the inner side thereof, and the second blade includes an arcuate slot formed in the inner side thereof for receiving the protrusion of the first blade, and wherein relative rotation between the first and second blades is limited by an arc length of the arcuate slot.

5. The replaceable blade assembly of claim 1, wherein the retainer includes a c-shaped body arranged within the axially-aligned axle openings and an enlarged radial shoulder arranged at a first end of the c-shaped body that abuts the outer side of the first blade.

6. The replaceable blade assembly of claim 5, further comprising a c-shaped spacer arranged at a second end of the c-shaped body that abuts the outer side of the second blade.

7. The replaceable blade assembly of claim 1, wherein a width of the proximal end of each blade decreases towards the proximal end.

8. The replaceable blade assembly of claim 1, wherein the open-ended slot includes a constricted portion positioned between the axle slot and the axle opening, and wherein the slot defines a gap that is smallest at the constricted portion.

9. A surgical tool, comprising:
a drive housing;
an elongate shaft extending distally from the drive housing;
a wrist arranged at a distal end of the shaft and including:
a clevis providing a main body and opposing first and second arms extending distally from the main body;
an opening defined in each arm; and
an axle mounted to the clevis and extending through each opening; and
an end effector operatively coupled to the wrist and including:
first and second blade holders rotatably mounted to the axle and arranged between the first and second arms; and
a blade assembly removably mounted to the axle and including a first blade received at the first blade holder, and a second blade received at the second blade holder,
wherein the first and second blades each include an open-ended slot provided at a proximal end of the first and second blades, and wherein the blade assembly is rotatably mounted to the axle at the open-ended slot.

10. The surgical tool of claim 9, wherein the first and second blades each include opposing inner and outer sides, and the open-ended slot provides an axle slot that extends into an axle opening, the blade assembly further comprising:
a retainer arranged through axially-aligned axle openings of the open-ended slots and operable to couple the first and second blades together with the inner side of each blade facing each other and while permitting relative rotation between the first and second blades.

11. The surgical tool of claim 10, wherein the retainer includes a c-shaped body arranged within the axially-aligned axle openings and an enlarged radial shoulder arranged at a first end of the c-shaped body that abuts the outer side of the first blade.

12. The surgical tool of claim 11, further comprising a c-shaped spacer arranged at a second end of the c-shaped body that abuts the outer side of the second blade.

13. The surgical tool of claim 9, wherein the first blade and the second blade each include a boss extending from the outer side, and the first and second blade holders each include an engagement slot for receiving the respective boss when assembled, wherein bosses are keyed to their respective engagement slot.

14. The surgical tool of claim 9, wherein the first blade includes a projection extending from the inner side thereof, and the second blade includes an arcuate slot formed in the inner side thereof for receiving the protrusion of the first blade, wherein relative rotation between the first blade and the second blade is limited by an arc length of the arcuate slot.

15. A method of installing a blade assembly within an end effector of a surgical tool, comprising:
advancing a proximal end of the blade assembly toward the end effector, the end effector including first and second blade holders rotatably mounted to an axle mounted to a clevis of a wrist of the surgical tool;
inserting the proximal end of the blade assembly into an insertion slot cooperatively defined by the first and second blade holders; and
removably mounting the blade assembly on the axle within the insertion slot, the blade assembly including a first blade engageable with the first blade holder, and a second blade engageable with the second blade holder,
wherein the first and second blades each include an open-ended slot provided at the proximal end, and wherein the removable blade assembly is rotatably mounted to the axle at the open-ended slot.

16. The method of claim 15, further comprising removing the blade assembly from the end effector by dislodging the blade assembly from the axle and withdrawing the blade assembly from the insertion slot.

17. The method of claim 15, further comprising seating a first boss extending from the outer side of the first blade within an engagement slot formed in the first blade holder and seating a second boss extending from the outer side of the second blade within an engagement slot formed in the second blade holder.

18. The method of claim 17, wherein bosses are keyed to their respective engagement slot.

19. The method of claim 15, further comprising limiting relative rotation between the first blade and the second blade via a projection extending from the inner side of the first blade and an arcuate slot formed in the second blade for receiving the protrusion of the first blade, wherein an amount of relative rotation between the first blade and the second blade is defined by an arc length of the arcuate slot.

20. The method of claim 15, wherein a bushing is arranged on the axle and the blade assembly further includes a retainer arranged through axially-aligned axle openings of the open-ended slots for coupling the first and second blades together with the inner side of each blade facing each other and while permitting relative rotation between the first and second, and wherein the method further includes: seating the retainer on the bushing.
